(19) United States
(12) Patent Application Publication
(10) Pub. No.: US 2018/0369298 A1
(43) Pub. Date: Dec. 27, 2018

(54) AN AGENT FOR PROMOTING THE GROWTH OF, AND/OR SUPPRESSING A DECREASE IN, BIFIDOBACTERIUM BACTERIA AND/OR LACTIC ACID BACTERIA

(71) Applicant: MORINAGA MILK INDUSTRY CO., LTD., Tokyo (JP)

(72) Inventors: Kengo FUJII, Zama-shi (JP); Hirofumi MIYAUCHI, Zama-shi, Kanagawa (JP); Yohei SATO, Zama-shi, Kanagawa (JP)

(73) Assignee: MORINAGA MILK INDUSTRY CO., LTD., Tokyo (JP)

(21) Appl. No.: 16/063,630

(22) PCT Filed: Dec. 19, 2016
(86) PCT No.: PCT/JP2016/087751

§ 371 Date: Jun. 18, 2018

(30) Foreign Application Priority Data
Dec. 18, 2015 (JP) 2015-247921

(51) Int. Cl. A61K 35/745; A61K 35/748; A61K 47/26; A61P 1/12; A61P 3/04

(52) U.S. Cl. CPC A61K 35/745; A61K 35/748; A61K 47/42; A61P 1/12; A61P 3/04; A61K 47/26

(57) ABSTRACT

To provide a technology capable of promoting growth of Bifidobacterium bacteria and lactic acid bacteria under an environment causing the growth or no change in cell count while suppressing decrease under an environment causing the decrease. Provided is an agent for promoting the growth of, and/or suppressing a decrease in, Bifidobacterium bacteria and/or lactic acid bacteria containing Arthrospira blue-green algae and/or Spirulina blue-green algae as an active ingredient. Also provided is an oral composition including Arthrospira blue-green algae and/or Spirulina blue-green algae together with at least one type of bacteria selected from the group consisting of Bifidobacterium bacteria and lactic acid bacteria. Furthermore provided is a method for promoting growth of Bifidobacterium bacteria and/or lactic acid bacteria and/or suppressing the decrease including a step for allowing at least one type of bacteria selected from the group consisting of Bifidobacterium bacteria and lactic acid bacteria to coexist with Arthrospira blue-green algae and/or Spirulina blue-green algae.

### TECHNICAL FIELD

The present technology relates to a growth-promoting and/or decrease-suppressing agent used for promoting growth of, or suppressing decrease in, Bifidobacterium bacteria (referred to also as “bifidobacteria”) and/or lactic acid bacteria, as well as a method for promoting the growth and/or suppressing the decrease. Also, the present technology relates to an oral composition including Bifidobacterium bacteria and/or lactic acid bacteria together with the relevant growth-promoting and/or decrease-suppressing agent.

### BACKGROUND ART

Bifidobacterium bacteria are enteric microorganisms in mammals (including human) and known to have no pathogenicity themselves and, in favor of their lactic acid production and auxotrophy, rather act antagonistically on pathogenic intestinal bacteria, the growth of which is thereby inhibited in the intestines.

Generally, the intestinal flora in an infantile large intestine is dominated by Bifidobacterium bacteria, but the flora is changed along with aging. Typically, Bifidobacterium bacteria decrease over a period of adolescent to mature ages, while putrefactive bacteria such as Clostridium bacteria or Escherichia coli increase markedly. As the result, the intestinal environment is deteriorated and affects the health of the host (human) adversely. Accordingly, it is extremely important to keep the intestinal flora in a condition allowing useful bacteria such as Bifidobacterium bacteria to be always dominant in order to keep the health of a human in a good condition even when aged.

Meanwhile, lactic acid bacteria are usually contained in dairy foods such as yogurts or fermented foods such as pickles, which are ingested by mammals (including human) thereby being introduced into intestines where they normalize the intestinal bacteria balance or exert their intestinal function-regulating effects. Recently, it was also indicated that the lactic acid bacteria serve to suppress allergic diseases such as hay fever, atopic dermatitis, and asthma and are expected to be effective in suppressing blood HDL cholesterol reduction and lowering neutral fat level.

Under such a circumstance, a substance which promotes the growth of Bifidobacterium bacteria and lactic acid bacteria (hereinafter referred to also simply as “growth promoter”) was demanded, and a large number of growth promoters were developed and proposed.

Currently known Bifidobacterium bacteria growth promoters may be oligosaccharides such as fructo-oligosaccharides, galacto-oligosaccharides, xylo-oligosaccharides, isomalto-oligosaccharides, and soybean oligosaccharides; saccharides such as N-acetylglucosamine, lactulose, raffinose, theanderose, cyclodextrin, and glucomannan (all so far in Patent Documents 1 to 4, Non-Patent Documents 1 to 3); soy milk and soy milk extracts (Patent Documents 5 and 6); tea leaf extracts (Patent Document 7); calcium phosphate (Patent Document 8); and bovine lactoferrin, bovine apo-lactoferrin, and iron bovine lactoferrin (Patent Document 9).

### CITATION LIST

### Patent Documents

[Patent Document 1] JP-A No. S60-41449
[Patent Document 2] JP-A No. H03-183454
[Patent Document 3] JP-A No. S57-138385
[Patent Document 4] JP-A No. H10-175867
[Patent Document 5] JP-B No. S45-9822
[Patent Document 6] JP-A No. S59-179064
[Patent Document 7] JP-A No. H01-191680
[Patent Document 8] JP-A No. 2005-130804
[Patent Document 9] JP-A No. H08-38044

### Non Patent Documents

[Non-Patent Document 1] “Bifidobacteria”, p. 77, 1979, Yakult Honsha Co., Ltd.
[Non-Patent Document 2] “Chemistry and Biology”, vol. 21, p. 291, 1983, Gakkai Shuppan Center
[Non-Patent Document 3] “RIKEN Intestinal Flora Symposium, Intestinal flora and Nutrition”, p. 89, 1983, Gakkai Shuppan Center

### SUMMARY OF THE INVENTION

### Technical Problem

As described above, a further development of substances which promote growth of Bifidobacterium bacteria and lactic acid bacteria was demanded continuously. Bifidobacterium bacteria and lactic acid bacteria exhibit promoted growth, no change, or decrease in cell counts depending on the environment where they exist.

Accordingly, a major object of the present technology is to provide a technology capable of promoting growth of Bifidobacterium bacteria and lactic acid bacteria under an environment causing the growth or no change in cell count while suppressing decrease under an environment causing the decrease.

### Solution to Problem

As a result of our intensive study to solve the aforementioned problem, it was discovered that coexistence of blue-green algae belonging to the genus of Arthrospira or Spirulina with Bifidobacterium bacteria and/or lactic acid bacteria results in a significant promotion of the growth of the latter thereby increasing the cell count, i.e., that Spirulina has a growth promoting effect on Bifidobacterium bacteria and/or lactic acid bacteria.

Thus, the present technology firstly provides an agent for promoting the growth of, and/or suppressing a decrease in, Bifidobacterium bacteria and/or lactic acid bacteria containing Arthrospira blue-green algae and/or Spirulina blue-green algae as an active ingredient.

The growth-promoting and/or decrease-suppressing agent according to the present technology may further contain at least one selected from the group consisting of proteins and saccharides.

In such a case, milk proteins and/or plant proteins can be selected as the aforementioned proteins.

As the aforementioned saccharides, at least one selected from the group consisting of monosaccharides, disaccharides, and oligosaccharides can be selected.

The present technology secondly provides an oral composition including Arthrospira blue-green algae and/or Spirulina blue-green algae, and at least one type of bacteria selected from the group consisting of Bifidobacterium bacteria and lactic acid bacteria.

The oral composition according to the present technology can further contain at least one selected from the group consisting of proteins and saccharides.

In such a case, milk proteins and/or plant proteins can be selected as the aforementioned proteins.

As the aforementioned saccharides, at least one selected from the group consisting of monosaccharides, disaccharides, and oligosaccharides can be selected.

Furthermore, the aforementioned oral composition can be used for intestinal flora improvement, immunoregulation, prophylaxis and/or treatment of diarrhea, constipation, obesity, or inflammatory bowel disease.

The present technology also provides a method for promoting growth of Bifidobacterium bacteria and/or lactic acid bacteria and/or suppressing the decrease including a step for allowing at least one type of bacteria selected from the group consisting of Bifidobacterium bacteria and lactic acid bacteria to coexist with Arthrospira blue-green algae and/or Spirulina blue-green algae.

In the method for promoting growth and/or suppressing decrease according to the present technology, it is also possible to allow at least one selected from the group consisting of proteins and saccharides to coexist.

In such a case, milk proteins and/or plant proteins can be selected as the aforementioned proteins.

As the aforementioned saccharides, at least one selected from the group consisting of monosaccharides, disaccharides, and oligosaccharides can be selected.

### Advantageous Effects of Invention

The present technology is capable of promoting growth of Bifidobacterium bacteria and lactic acid bacteria under an environment causing the growth or no change in cell count while suppressing decrease under an environment causing the decrease. The effects described here are not necessarily limitative, and any of the effects described in the present technology is contemplated.

### DESCRIPTION OF EMBODIMENTS

The followings are the descriptions of preferred embodiments for implementing the present technology. The embodiments described below indicate examples of the representative embodiments of the present disclosure, which do not allow the scope of the present technology to be interpreted to be narrower. When a range of numerical values is expressed as “lower limit to upper limit” in the present specification, the upper limit may be preceded by “not more than” or “less than” while the lower limit may be preceded by “not less than” or “exceeding”.

### 1. Growth-Promoting and/or Decrease-Suppressing Agent

A growth-promoting and/or decrease-suppressing agent according to the present technology (hereinafter referred to also simply as “growth-promoting/decrease-suppressing agent”) is a formulation containing Arthrospira blue-green algae and/or Spirulina blue-green algae as an active ingredient, and characterized by its action effect which promotes the growth of at least one type of bacteria selected from the group consisting of Bifidobacterium bacteria and lactic acid bacteria (hereinafter referred to also simply as “Bifidobacterium bacteria and/or lactic acid bacteria” or “Bifidobacterium bacteria/lactic acid bacteria”). It is also possible that the growth promoter of the present technology may contain at least one selected from the group consisting of proteins and saccharides in addition to Arthrospira blue-green algae and/or Spirulina blue-green algae.

The followings are a detailed description of the present technology.

### (1) Bifidobacterium Bacteria

Those included by the present technology as Bifidobacterium bacteria may for example be Bifidobacterium lactis, Bifidobacterium longum, Bifidobacterium infantis, Bifidobacterium adolescentis, Bifidobacterium breve, Bifidobacterium bifidum, and Bifidobacterium animalis. These Bifidobacterium bacteria may be subjected independently or in combination of any two or more. Those employed preferably may for example be, but are not limited to, Bifidobacterium longum, Bifidobacterium breve, and Bifidobacterium infantis. Those employed more preferably are Bifidobacterium longum and Bifidobacterium breve.

### (2) Lactic Acid Bacteria

Those included by the present technology as lactic acid bacteria may for example be Lactococcus lactis subsp. lactis, Lactococcus lactis subsp. cremoris (so far Lactococcus bacteria); Streptococcus salivarius subsp. thermophilus (so far Streptococcus bacteria, referred to also as Enterococcus bacteria); Leuconostoc mesenteroides subsp. cremoris (so far Leuconostoc bacteria); Lactobacillus acidophilus, Lactobacillus casei, Lactobacillus delbrueckii subsp. bulgaricus, Lactobacillus delbrueckii subsp. lactis, Lactobacillus gasseri, Lactobacillus helveticus, Lactobacillus plantarum, Lactobacillus brevis, Lactobacillus casei subsp. rhamnosus, Lactobacillus paracasei (so far Lactobacillus bacteria); Tetragenococcus halophilus (so far Tetragenococcus bacteria) and Pediococcus pentosaseus (so far Pediococcus bacteria). These lactic acid bacteria may be subjected independently or in combination of any two or more. Those employed preferably may for example be, but are not limited to, Lactobacillus gasseri, Lactobacillus acidophilus, and Lactobacillus paracasei. More preferably, Lactobacillus gasseri is employed.

### (3) Arthrospira Blue-Green Algae and/or Spirulina Blue-Green Algae (Hereinafter Referred to Also as “Spirulina”)

Spirulina includes blue-green algae belonging to the genus of Arthrospira or Spirulina. Those included in such blue-green algae may for example be Arthrospira (Spirulina) platensis, Arthrospira (Spirulina) maxima, Spirulina subsalsa, Spirulina major, Spirulina geitleri, Spirulina siamese, Spirulina princeps, Spirulina laxissima, Spirulina curta, Spirulina spirulinoides, and Spirulina aldaria. Those included in Spirulina may be employed independently or in combination of any two or more.

Spirulina is known to contain a large amount of minerals such as calcium (400 to 1200 mg: amount contained in 100 g of Spirulina, the same applies below), potassium (800 to 2000 mg), iron (50 to 150 mg), zinc (1 to 3 mg), and copper (0.3 to 0.6 mg) in addition to proteins, chlorophyll, carotenes, and vitamin Bs (see for example, Ikuo Saiki “Kyukyokuno Kanzenshokuhin (Ultimately perfect food) Spirulina”, Takanawa-Shuppansha, Dec. 20, 1996 pp. 62-65).

The form of the Spirulina to be incorporated into a growth-promoting/decrease-suppressing agent according to the present technology is not limited particularly, and may be an algae body as being cultured in a liquid medium (referred to also as “wet algae body”), or algae body debris, for example, ultrasonicated debris or homogenizer-pulverized debris, and a dry algae body obtained therefrom by drying. Such a dry algae body may be a powder formulation obtained by milling. The form of a dry algae body is preferred, and an algae body as a dried powder is more preferred. While a dry algae body of Spirulina can be prepared by drying Spirulina which was obtained by a private cultivation (see for example Japanese Unexamined Patent Application Publication No. 2006-25668), it is available also commercially (for example from Spirulina Bio-Lab Co., Ltd.).

Another form of Spirulina to be incorporated into a growth-promoting/decrease-suppressing agent according to the present technology may be a solvent extract of Spirulina algae body or wet algae body obtained by using water, hot water, organic solvents and the like. Various extracts can be obtained by extraction methods such as subjecting Spirulina algae body or wet algae body to a pulverization of such an algae body or a part thereof preferably using homogenizer, ultrasonic pulverization and the like to obtain a liquefied material, which is then extracted with a solvent such as water, hot water, and organic solvents. More preferably, a method may be exemplified in which 1 part by mass of Spirulina algae body or wet algae body was combined with 1 to 500 parts by mass of the aforementioned solvent and refluxed with stirring or shaking at a temperature not higher than the boiling point of the solvent or in which ultrasonic extraction is conducted at ambient temperature.

The extract solution can be subjected to a suitable method for separating insolubles such as filtration or centrifugation to obtain a crude extract.

The aforementioned organic solvent may for example be alcohols such as methanol, ethanol, and butanol; esters such as methyl acetate, ethyl acetate, propyl acetate, and butyl acetate; ketones such as acetone and methyl isobutyl ketone; ethers such as diethyl ether and petroleum ether; hydrocarbons such as hexane, cyclohexane, toluene, and benzene; halogenated hydrocarbons such as carbon tetrachloride, dichloromethane, and chloroform; heterocyclic compounds such as pyridine; glycols such as ethylene glycol; polyalcohols such as polyethylene glycol; and nitrile solvents such as acetonitrile as well as mixtures of such solvents. These solvents may be anhydrous or hydrated.

The growth-promoting/decrease-suppressing agent according to the present technology may consist of 100% by mass of Spirulina, or can contain other components in addition to Spirulina as described above. Accordingly, the growth-promoting/decrease-suppressing agent according to the present technology can have a Spirulina content which can be appropriately adjusted within a range of 0.01 to 100% by mass.

The growth-promoting/decrease-suppressing agent according to the present technology can be employed in a ratio so that the final Spirulina concentration becomes 0.0001 to 100% by mass, preferably 0.1 to 100% by mass depending on the final form thereof such as medicaments or quasi-drugs or foods/beverages as described below.

In case of oral ingestion of a growth-promoting/decrease-suppressing agent according to the present technology for the purpose of growing Bifidobacterium bacteria in an in vivo intestine, an example of the amount ingested per day as a Spirulina quantity contained in the growth-promoting/decrease-suppressing agent according to the present technology is usually 10 mg to 20 g/day, preferably 30 mg to 10 g/day, more preferably 1 to 6 g/day. The relevant ingestion amount may be taken as a subdivided dose twice or more a day.

### (4) Proteins

As described above, in addition to Spirulina, proteins can also be incorporated into the growth-promoting/decrease-suppressing agent according to the present technology. By incorporating the proteins, Bifidobacterium bacteria and/or lactic acid bacteria growth-promoting and/or decrease-suppressing effects become more excellent as indicated in Examples described below.

The proteins which can be employed in the growth-promoting/decrease-suppressing agent according to the present technology may be any arbitrarily selected one or two or more proteins capable of being used in the field of medicines and foods. Those exemplified typically are milk-derived proteins such as caseins and whey proteins and plant-derived proteins such as soy proteins.

As caseins, various commercially available caseins and caseinates can for example be utilized. Those which can be exemplified more typically are lactic acid casein, sulfuric acid casein, hydrochloric acid casein, sodium caseinate, potassium caseinate, calcium caseinate, magnesium caseinate, or mixtures thereof. It is also possible to utilize caseins purified by standard methods from cow milk, skim milk, whole milk powder, and skim milk powder.

As whey proteins, commercially available products or whey separated by known method from cow milk, skim milk, and the like (for example, whey powder, desalted whey powder) or separated and purified whey protein concentrates, whey protein isolates, or mixtures thereof in any ratios can be employed.

While these proteins can be used as proteins in forms as being purified, they can be used also as raw material forms containing such proteins.

Raw materials containing proteins derived from milks may for example be raw milk, cow milk, buffalo milk, goat milk, sheep milk, horse milk, concentrated milk, skimmed concentrated milk, skim milk powder, whey protein concentrate (WPC), whey protein isolate (WPI), milk protein concentrate (MPC), micellar casein isolate (MCI), and milk protein isolate (MPI).

“Milk” as used herein in the present technology includes those prescribed under “Ministerial Ordinance on Milk and Milk products Concerning Compositional Standards, etc.” (hereinafter referred to also simply as “Ministerial Ordinance on Milks”) such as raw milk, cow milk, special cow milk, raw goat milk, pasteurized goat milk, partially skimmed milk, skim milk, and processed milk (so far all maternal milks of mammals) as well as plant-derived milk such as soy milk, almond milk, and coconut milk. While any of these can be independently incorporated into a growth promoter of the present technology, any combination of two or more can be incorporated. The aforementioned maternal milks of mammals and soy milk are preferred, and those more preferred are bovine raw milk, cow milk, special cow milk, partially skimmed milk, skim milk, and processed milk as well as soy milk. The definitions of the aforementioned “raw milk, cow milk, special cow milk, raw goat milk, pasteurized goat milk, partially skimmed milk, skim milk, and processed milk” are understood to be based on Ministerial Ordinance on Milks.

With regard to the composition of a cow milk on an average basis, the ingredients are proteins in an amount of 3.3% by mass, fats in an amount of 3.8% by mass, carbohydrates in an amount of 4.8% by mass, minerals (potassium, calcium, phosphates, magnesium, sodium, citrates, phosphorus, iron, and the like) in an amount of 0.37% by mass as well as vitamins (vitamins A, B1, B2, C, and E) and water, and the calorie per 100 grams is about 67 kilocalories. About 80% by mass of the proteins contained in a cow milk corresponds to caseins (casein micelle). The remainder 20% by mass corresponds to whey proteins, including proteins such as ß-lactoglobulin, α-lactoglobulin, immunoglobulin, serum albumin and lactoferrin. The carbohydrates contained in a cow milk may for example be lactose (disaccharide), glucose, galactose (both monosaccharides), and other oligosaccharides. The carbohydrates are mostly lactose, which corresponds to 50% by mass of the solid content of skim milk powder. The remainder 50% by mass of the skim milk powder (solid content) corresponds mostly to proteins.

Raw materials containing plant-derived proteins may for example be soy milk, almond milk, and coconut milk.

The soy milk is obtained by immersing soybeans in water, grinding, cooking with water to reduce the volume, and then filtering the resultant fluid.

The almond milk is obtained by pulverizing water-immersed almonds for example using a mixer, adding water, and filtering the cake off for example using a gauze.

The coconut milk is a sweet milk-like food material obtained from a solid endosperm formed laminarly inside of a mature coconut seed.

Also in a growth-promoting/decrease-suppressing agent according to the present technology, a product obtained by further processing a protein-containing raw material (for example a dairy product) can be incorporated.

A dairy product means an edible product obtained by processing the aforementioned milk (preferably raw milk and soy milk) artificially. For example, dairy products prepared from raw milks are creams, butter, butter oils, cheese, concentrated whey, ice cream products (ice cream, lacto-ice, ice-milk), concentrated milks, skimmed concentrated milks, non-sugar condensed milks, non-sugar skimmed condensed milks, sugar-added condensed milks, sugar-added skimmed condensed milks, whole milk powder, skim milk powder, cream powder, whey powder, butter milk powder, sugar-added milk powder, formulated milk powder, fermented milks, lactic acid bacteria drinks (limited to those having a non-fat milk solid content of 3% or more), and milk-based drinks. The definitions of the respective ingredients are understood to be based on Ministerial Ordinance on Milks. Among these, skim milk (whey, concentrated whey, cottage cheese, skimmed concentrated milk, non-sugar skimmed condensed milk, sugar-added skimmed condensed milk, skimmed concentrated milk, non-sugar skimmed condensed milk, sugar-added skimmed condensed milk, skim milk powder, whey powder), milk powder (whole milk powder, skim milk powder, cream powder, whey powder, butter milk powder, sugar-added milk powder, formulated milk powder) are preferred, and skim milk powder and whey powder are more preferred.

When incorporating a protein to a growth-promoting/decrease-suppressing agent according to the present technology, the amount to be incorporated is not limited particularly, and can arbitrarily be selected depending on the amount or the purpose of use of Spirulina. Especially in the present technology, it is preferable to incorporate 1 to 10000 parts by mass to 100 parts by mass of Spirulina, and it is more preferable to incorporate 50 to 300 parts by mass.

### (5) Saccharides

As described above, saccharides can be incorporated to a growth-promoting/decrease-suppressing agent according to the present technology in addition to Spirulina. By incorporating the saccharides, Bifidobacterium bacteria and/or lactic acid bacteria growth-promoting and/or decrease-suppressing effects become more excellent as indicated in Examples described below.

Saccharides which can be used in a growth-promoting/decrease-suppressing agent according to the present technology are arbitrarily selected one or two or more of saccharides capable of being used in the field of medicines and foods. Those which can typically be exemplified are monosaccharides such as glucose, fructose, galactose, rhamnose, and fucose; disaccharides such as lactose, sucrose, maltose, and trehalose; trisaccharides such as raffinose; oligosaccharides such as lactulose, fructo-oligosaccharides, galacto-oligosaccharides, mannan oligosaccharides, isomalto-oligosaccharides, and xylo-oligosaccharides; and polysaccharides such as N-acetylglucosamine, dextrin, and soybean oligosaccharides. Those preferred are monosaccharides, disaccharides, and oligosaccharides, and those more preferred are glucose and lactose. The lactulose is obtained by subjecting lactose to an alkali isomerization by a known method and is commercially available, while it can be produced according to the method described in Japanese Examined Patent Application Publication No. S52-21063.

When incorporating a saccharide to a growth-promoting/decrease-suppressing agent according to the present technology, the amount to be incorporated is not limited particularly, and can arbitrarily be selected depending on the amount or the purpose of use of Spirulina. Especially in the present technology, it is preferable to incorporate 1 to 20000 parts by mass to 100 parts by mass of Spirulina, and it is more preferable to incorporate 80 to 500 parts by mass.

### (6) Other Ingredients

In a growth-promoting/decrease-suppressing agent according to the present technology, arbitrarily selected one or two or more other ingredients capable of being used in the field of medicines and foods can be used as long as the effects of the present technology are not affected adversely.

Such other ingredients may for example be carriers and additives used for formulating Spirulina. Typically, ingredients such as excipients, pH modifiers, colorants, and taste modifiers can be used. Another option is concomitant use of ingredients having prophylactic, ameliorating, and/or therapeutic effects on diseases or symptoms which are known or will be found in future appropriately depending on the purpose.

The aforementioned formulation carrier may be any of various organic or inorganic carriers depending on dosage forms. In a solid formulation, the carrier may for example be an excipient, binder, disintegrant, lubricant, stabilizer, and flavoring agent.

The aforementioned excipient may be saccharide derivatives such as lactose, sucrose, glucose, mannitol, and sorbitol; starch derivatives such as corn starch, potato starch, α-starch, dextrin, and carboxymethyl starch; cellulose derivatives such as crystalline cellulose, hydroxypropyl cellulose, hydroxypropyl methyl cellulose, carboxymethyl cellulose, and carboxymethyl cellulose calcium; gum arabic; dextran; pullulan; silicate derivatives such as light silicic anhydride, synthetic aluminum silicate, and magnesium aluminometasilicate; phosphate derivatives such as calcium phosphate; carbonate derivatives such as calcium carbonate; and sulfate derivatives such as calcium sulfate.

The aforementioned binder may for example be gelatin; polyvinyl pyrrolidone; and Macrogol, in addition to any of the excipients described above.

The aforementioned disintegrant may for example be a chemically modified starches or cellulose derivatives such as croscarmellose sodium, carboxymethyl starch sodium, and crosslinked polyvinyl pyrrolidone in addition to any of the excipients described above.

The aforementioned lubricant may for example be talc; stearic acid; metal stearates such as calcium stearate, and magnesium stearate; colloidal silica; waxes such as veegum and spermaceti; boric acid; glycol; carboxylic acids such as fumaric acid and adipic acid; sodium carboxylates such as sodium benzoate; sulfates such as sodium sulfate; leucine; lauryl sulfates such as sodium lauryl sulfate and magnesium lauryl sulfate; silicic acids such as silicic anhydride and silicic hydrate; and starch derivatives.

The aforementioned stabilizer may for example be p-hydroxybenzoates such as methyl paraben and propyl paraben; alcohols such as chlorobutanol, benzyl alcohol, and phenylethyl alcohol; benzalkonium chloride; acetic anhydride; and sorbic acid.

The aforementioned flavoring agent may for example be sweeteners, acidifiers, and flavors.

The carrier employed in a liquid formulation for oral administration may for example be water and flavoring agents.

### (7) Use

The growth-promoting/decrease-suppressing agent according to the present technology is employed mainly for the purpose of promoting the growth of Bifidobacterium bacteria/lactic acid bacteria or suppress the decrease, thereby increasing the cell counts of the relevant bacteria or thereby improving the viability of the relevant bacteria.

As used in the present technology, “growth promotion” (growth-promoting effect) means an effect on Bifidobacterium bacteria/lactic acid bacteria (viable bacteria) under an environment enabling a survival thereby promoting the growth thereof.

As used herein the present technology, “decrease suppression” (decrease-suppressing effect) means to allow the relevant Bifidobacterium bacteria/lactic acid bacteria to undertake a growth promotion under an environment which suppresses survival or development (growth) thereby suppressing the decrease in the viable bacterial count due to death (inactivation) and maintaining or increasing the count of bacteria which remain viable (viable bacterial count) (survival rate improvement).

Such growth-promoting effect and decrease-suppressing effect possessed by a growth-promoting/decrease-suppressing agent according to the present technology can be judged using an index which is a higher survival rate (%) calculated according to the equation shown below based on the viable bacterial counts of Bifidobacterium bacteria/lactic acid bacteria before and after the culture when Bifidobacterium bacteria/lactic acid bacteria (viable bacteria) are cultured in the presence of the growth-promoting/decrease-suppressing agent according to the present technology when compared with the survival rate (%) calculated when Bifidobacterium bacteria/lactic acid bacteria (viable bacteria) are cultured in the absence of the growth-promoting/decrease-suppressing agent according to the present technology (control survival rate). For typical method and condition of the culture of Bifidobacterium bacteria/lactic acid bacteria, the methods described below in Experimental Examples can be referred to.

Survival rate (%)=[viable bacterial count after culture/viable bacterial count before culture]×100 [Equation 1]

When calculating the survival rate (%), the method for obtaining the viable bacterial count of Bifidobacterium bacteria/lactic acid bacteria is in accordance with a standard method. The method for measuring the viable bacterial count may for example be a colony counting method using TOS propionate acid agar medium (Yakult Pharmaceutical Industry Co., Ltd.) as a Bifidobacterium bacteria assay. The lactic acid bacteria measurement method may for example be a colony counting method using BCP-supplemented plate count agar medium “EIKEN” (Eiken Chemical Co., Ltd.).

Since a growth-promoting/decrease-suppressing agent according to the present technology is useful when applied to intestinal flora improvement, immunoregulation, prophylaxis and/or treatment of diarrhea, constipation, obesity, or inflammatory bowel disease and the like via Bifidobacterium bacteria/lactic acid bacteria growth promotion or decrease suppression, it can be used in the forms of medicaments or quasi-drugs, foods/beverages, feeds and the like for the aforementioned applications.

### (7-1) Medicaments and Quasi-Drugs

A growth-promoting/decrease-suppressing agent according to the present technology can be used as being incorporated as an active ingredient in a human or veterinarian medicament, quasi-drug, and the like utilizing its excellent Bifidobacterium bacteria/lactic acid bacteria growth-promoting/decrease-suppressing effect.

In the case of incorporation into a medicament, the medicament can be formulated into an appropriate and desired dosage form depending on the administration method such as oral administration or parenteral administration. While such a dosage form is not limited, the oral administration can employ formulations including solid formulations such as powder formulations, granule formulations, tablets, troches, capsules; and liquid formulations such as solution formulations, syrups, suspensions, and emulsions. The parenteral administration can employ formulations such as suppositories, spray formulations, inhalation formulations, ointments, patches, and injection formulations. In the present disclosure, formulation into a dosage form for oral administration is preferred.

The formulation can be implemented appropriately by known methods depending on dosage forms.

### (7-2) Foods/Beverages

A growth-promoting/decrease-suppressing agent according to the present technology can be used as being incorporated as an active ingredient in a human or veterinarian health food, functional food, patient food, enteral nutrition food, food for special use, food with health claims, food for specified health uses, food with function claims, nutritional functional food and the like utilizing its excellent Bifidobacterium bacteria/lactic acid bacteria growth-promoting/decrease-suppressing effect.

A growth-promoting/decrease-suppressing agent according to the present technology can be added to a known food/beverage during the preparation or can also be mixed into the raw materials of the food/beverage to produce a new food/beverage.

The aforementioned foods/beverages, regardless of their forms such as liquids, pastes, solids, and powders, may for example be tablet confectioneries and liquid diets, as well as flour products, instant foods, processed agricultural products, processed marine products, processed livestock products, milk and dairy products, fats, basic seasonings, composite seasoning or food products, frozen foods, confectioneries, beverages, other commercially available products.

The aforementioned flour products may for example be breads, macaronis, spaghetties, noodles, cake mixes, deep frying flours, and bread crumbs.

The aforementioned instant foods may for example be instant noodles, cup-contained instant noodles, retort-cooked foods, cooked and canned foods, microwaving foods, instant soups or stews, instant miso soups or clear soups, canned soups, freeze-dried foods, and other instant foods.

The aforementioned processed agricultural products may for example be canned agricultural products, canned fruits, jams or marmalades, pickles, boiled beans, agricultural dry foods, and cereals (processed grain products).

The aforementioned processed marine products may for example be canned marine products, fish meat hams and sausages, marine paste products, marine delicacies, and cocked and seasoned “tsukudani” foods.

The aforementioned processed livestock products may for example be canned livestock/paste products and livestock meat hams and sausages.

The aforementioned milk and dairy products may for example be processed milks, milk beverages, yogurt foods, fermented milk beverages, cheeses, ice creams, formulated milk powders, creams, and other dairy products.

The aforementioned fats may for example be butters, margarines, and vegetable oils.

The aforementioned basic seasonings may for example be soy sauces, misos, sauces, processed tomato seasonings, fermented seasoning “mirin” products, and vinegars, and the aforementioned composite seasoning or food products may for example be cooking mixes, curry bases, sauces, dressings, noodle soup bases “mentsuyu”, spices, and other composite seasonings.

The aforementioned frozen foods may for example be material frozen foods, half-cooked frozen foods, and cooked frozen food.

The aforementioned confectioneries may for example be caramels, candies, chewing gums, chocolates, cookies, biscuits, cakes, pies, snacks, crackers, Japanese sweets, rice confectioneries, bean confectioneries, desserts, and other confectioneries.

The aforementioned beverages may for example be carbonated drinks, natural fruit juices, fruit juice drinks, fruit juice-containing soft drinks, fruit pulp drinks, granule-containing fruit drinks, vegetable-based beverages, soy milks, soy milk beverages, coffee beverages, tea beverages, powdered beverages, concentrated drinks, sports drinks, nutritional drinks, alcoholic drinks, and other tasty beverages.

Other commercial foods may for example be baby foods, dried seasoning powders “furikake”, and dried seasoning powders “ochazukenori”.

On the other hand, the foods/beverages defined in the present disclosure can be provided or sold as foods/beverages which claim specific use (especially health use) or functions.

The action of “claiming” includes every action for making a consumer to be informed of the aforementioned use, and any expression, which reminds the consumer of, or, which allows the consumer to assume, the aforementioned use is regarded as the action of “claiming” regardless of the purpose of the claiming, the contents of the claiming, or the claimed subjects or media.

The “claiming” is implemented preferably via an expression by which the consumer can recognize the aforementioned use directly. Those which may be typically exemplified include an activity to assign, deliver, display for the purpose of assignment or delivery and export a good or good package relating to a food or drink having a description of the aforementioned use thereon, as well as an activity to display or distribute advertisement materials, price lists, or transaction documents relating to goods having a description of the aforementioned use thereon or to provide such a detailed information also including a description of the aforementioned use therein via an electromagnetic method (such as internet).

Meanwhile, the claimed detail is preferably one which has been authorized by the relevant government (for example the claim was authorized under various regulations prescribed by the government and was implemented in a manner based on such an authorization). Such a claimed detail is preferably attached to the advertising materials at the site of selling such as a package, container, catalog, pamphlet, and POP as well as other documents.

The “claiming” may also be a claiming as a health food, functional food, patient food, enteral nutrition food, food for special use, food with health claims, food for specified health uses, food with function claims, nutritional functional food, and quasi-drug. Among these, one which may especially be exemplified is a claim authorized by Consumer Affairs Agency, for example, a claim authorized under the regulation on food for specified health uses, the regulation on food with function claims, or analogous regulations. Those exemplified more typically are a claim as a food for specified health uses, a claim as a conditional food for specified health uses, a claim as a food with function claims, a claim claiming an effect on body structure or function, and a disease risk reduction claim. Among these, those exemplified more typically are a claim as a food for specified health uses (especially health use claim) prescribed under the Ordinance for Enforcement of Health Promotion Act (Ordinance of the Ministry of Health, Labour and Welfare No. 86 dated Apr. 30, 2003), a claim as a food with function claims prescribed under Food Labeling Act (Act No. 70 of 2013), and analogous claims.

### (7-3) Feeds

A growth-promoting/decrease-suppressing agent according to the present technology can be used as an active ingredient in a veterinarian feed utilizing its excellent Bifidobacterium bacteria/lactic acid bacteria growth-promoting/decrease-suppressing effect. A growth-promoting/decrease-suppressing agent according to the present technology can be added to a known feed during the preparation or can also be mixed into the raw materials of the feed to produce a new feed.

The raw materials for the aforementioned feeds may be cereals such as corn, wheat, barley, and rye; brans such as wheat bran, oat bran, rice bran, and skimmed rice bran; food manufacturer's by-products such as corn gluten meal and corn jam meal; animal-derived feeds such as skim milk powder, whey, fish meal, and born meal; yeasts such as brewer's yeast; mineral feeds such as calcium phosphate and calcium carbonate; oils and fats; amino acids; and saccharides. Examples of the aforementioned feeds may be feeds for a companion animal (pet food and the like), livestock feed, and fish breeding feed.

### 2. Oral Compositions

The oral composition according to the present technology is a composition at least including Spirulina and at least one type of bacteria selected from the group consisting of Bifidobacterium bacteria and lactic acid bacteria. If necessary, at least one selected from the group consisting of proteins and saccharides may be contained. The aforementioned oral composition can be used for intestinal flora improvement, immunoregulation, prophylaxis and/or treatment of diarrhea, constipation, obesity, or inflammatory bowel disease and the like.

The amount of Bifidobacterium bacteria/lactic acid bacteria in an oral composition is not limited particularly, and can arbitrarily be selected depending on the purpose of use and the like. Especially in the present technology, 1 g of the oral composition can contain Bifidobacterium bacteria and/or lactic acid bacteria (total amount when containing the both) usually at 1×10⁴ cfu/g or more, preferably within the range from 1×10⁶ to 1×10¹² cfu/g.

With regard to the details of Bifidobacterium bacteria/lactic acid bacteria, Spirulina, proteins, saccharides, and other ingredients, no description was made here since they are identical to those in the aforementioned growth-promoting and/or decrease-suppressing agent.

### 3. Bifidobacterium Bacteria/Lactic Acid Bacteria Growth Promoting and/or Decrease Suppressing Method

A Bifidobacterium bacteria/lactic acid bacteria growth promoting and/or decrease suppressing method of the present technology (hereinafter referred to also simply as “growth-promoting/decrease-suppressing method”) is a method including a step for allowing at least one type of bacteria selected from the group consisting of Bifidobacterium bacteria and lactic acid bacteria to coexist with Spirulina. Also if necessary, it is possible to further allow at least one selected from the group consisting of proteins and saccharides to coexist.

With regard to the details of Bifidobacterium bacteria/lactic acid bacteria, Spirulina, proteins, saccharides, and other ingredients, no description was made here since they are identical to those in the aforementioned growth-promoting and/or decrease-suppressing agent.

### EXAMPLE

The present technology is further detailed below based on the Examples. The Examples described below only exemplifies representative Examples of the present technology, and should not be understood to allow the scope of the present technology to be interpreted narrowly.

### Experimental Example 1

In Experimental Example 1, effects of Spirulina on Bifidobacterium bacteria growth property were investigated.

As Bifidobacterium bacteria, Bifidobacterium longum and Bifidobacterium breve were employed. More typically, Bifidobacterium longum employed was a microorganism which was deposited to American Type Culture Collection (ATCC) under a deposition number of “BAA-999” (Bifidobacterium longum ATCC BAA-999) and which can commercially be available (http://www.atcc.org/products/all/BAA-999.aspx), and Bifidobacterium breve employed was a microorganism which was deposited to National Institute of Technology and Evaluation (NITE) under a deposition number of “FERM BP-11175” (Bifidobacterium breve FERM BP-11175), and which can commercially be available (the same shall apply hereinafter).

Spirulina employed was Spirulina powder obtained from Spirulina Bio-Lab Co., Ltd.

### (1) Preparation of Each Experimental Material

### (1-1) Bifidobacterium Bacteria Dry Powder Preparation

The respective Bifidobacterium bacteria were inoculated to a culture medium containing proteins, amino acids, and saccharides, cultured at 32 to 41° C. for 5 to 24 hours, and thereafter cells (wet cells) were collected from the culture fluid by centrifugation. A freeze-dryer (manufactured by Kyowa Vacuum Engineering Co., Ltd.) was used to conduct freeze-drying of Bifidobacterium longum for 18 to 96 hours, Bifidobacterium breve for 120 hours, and the cell aggregate after freeze-drying was milled mechanically to obtain each Bifidobacterium bacteria dry powder (hereinafter referred to as “Bifidobacterium bacteria powder”).

### (1-2) Spirulina Solution Preparation

3 g of Spirulina powder was dissolved in 100 mL of tap water or cow milk to prepare a Spirulina solution, which was subjected to autoclave pasteurization at 90° C. for 10 minutes to achieve sterilization.

### (2) Experimental Method

To 10 mL of each Spirulina solution placed in a test tube (sterilized), each Bifidobacterium bacteria powder diluted with physiological saline at a cell count per 10 mL of 1.0×10⁹ cfu (1.0×10⁸ cfu/mL) was inoculated and cultured at 37° C. for 8 hours and 16 hours. After culturing, the cell count of the cultured material (cfu/mL) and pH were measured, compared with the cell count and pH before culturing, and effects of Spirulina on Bifidobacterium bacteria growth property were evaluated. Also for comparison, the aforementioned Spirulina solution was changed to tap water or cow milk, and the experiment was conducted similarly.

### (3) Results of Experiment

Examples 1 to 4 and Comparatives 1 to 4 gave the results shown in Table 1.

TABLE 1

| | | | | | pH after culture | | | | Cell count after culture | | | |
|---|---|---|---|---|---|---|---|---|---|---|---|---|
| | | | | | 8 h | | 16 h | | (cfu/ml)(TOS) | | | |
| | Bifido- | | | pH | | vs | | vs | 8 h | | 16 h | |
| | bacterium | | Base | before | | before | | before | Cell | Survival | Cell | Survival |
| | bacteria | Spirulina | solution | culture | pH | culture | pH | culture | count | rate | count | rate |
| Example 1 | longum | ∘ | tap water | 6.98 | 7.04 | 0.06 | 6.91 | −0.07 | 4.8E+07 | 48% | 3.9E+07 | 39% |
| Example 2 | longum | ∘ | cow milk | 6.69 | 5.17 | −1.52 | 4.42 | −2.27 | 1.3E+09 | 1330% | 2.4E+09 | 2385% |
| Example 3 | breve | ∘ | tap water | 7.07 | 6.89 | −0.18 | 6.72 | −0.35 | 1.2E+08 | 116% | 1.1E+08 | 108% |
| Example 4 | breve | ∘ | cow milk | 7.07 | 6.88 | −0.19 | 6.80 | −0.27 | 4.1E+09 | 4100% | 6.8E+09 | 6750% |
| Comparative 1 | longum | x | tap water | 8.36 | 7.75 | −0.61 | 7.78 | −0.58 | 1.4E+07 | 14% | 2.4E+06 | 2.4% |
| Comparative 2 | longum | x | cow milk | 6.57 | 6.31 | −0.26 | 5.90 | −0.67 | 8.2E+07 | 82% | 4.7E+07 | 47% |
| Comparative 3 | breve | x | tap water | 8.39 | 7.51 | −0.88 | 7.47 | −0.92 | 5.2E+07 | 52% | 1.5E+07 | 15% |
| Comparative 4 | breve | x | cow milk | 6.64 | 6.23 | −0.41 | 6.04 | −0.60 | 9.8E+07 | 98% | 7.3E+07 | 73% |

As evident from the comparison between Examples 1 and 3 as well as Comparatives 1 and 3, in any of Bifidobacterium bacteria, culture in the presence of Spirulina resulted in a bacterial growth promotion and an increase in survivability.

Also as indicated in Comparative 2 and Comparative 4, the cow milk had a Bifidobacterium bacteria survivability increasing effect, while the combination of cow milk with Spirulina resulted in a further remarkably promoted bacteria growth of Bifidobacterium bacteria, thereby increasing the cell count far more substantially.

### Experimental Example 2

In Experimental Example 2, effects of Spirulina on lactic acid bacteria growth property were investigated.

As lactic acid bacteria, Lactobacillus gasseri was employed. More typically, Lactobacillus gasseri employed was a microorganism which was deposited to National Institute of Technology and Evaluation under a deposition number of “NITE BP-01669”, and which can commercially be available (the same shall apply hereinafter).

### (1) Preparation of Each Experimental Material

### (1-1) Lactic Acid Bacteria Dry Powder Preparation

The lactic acid bacteria were inoculated to a culture medium containing proteins, amino acids, and saccharides, cultured at 32 to 41° C. for 5 to 24 hours, and thereafter cells (wet cells) were collected from the culture fluid by centrifugation. A freeze-dryer (manufactured by Kyowa Vacuum Engineering Co., Ltd.) was used to conduct freeze-drying for 18 to 96 hours, and the cell aggregate after freeze-drying was milled mechanically to obtain lactic acid bacteria dry powder (hereinafter referred to as “lactic acid bacteria powder”).

### (1-2) Spirulina Solution Preparation

3 g of Spirulina powder was dissolved in 100 mL of tap water or cow milk to prepare a Spirulina solution, which was subjected to autoclave pasteurization at 90° C. for 10 minutes to achieve sterilization.

### (2) Experimental Method

To 10 mL of each Spirulina solution placed in a test tube (sterilized), lactic acid bacteria powder diluted with physiological saline at a cell count per 10 mL of 1.0×10⁹ cfu (1.0×10⁸ cfu/mL) was inoculated, and cultured at 37° C. for 8 hours. After culturing, the cell count of the cultured material (cfu/mL) and pH were measured, compared with the cell count and pH before culturing, and effects of Spirulina on lactic acid bacteria growth property were evaluated. Also for comparison, the aforementioned Spirulina solution was changed to tap water or cow milk, and the experiment was conducted similarly.

### (3) Results of Experiment

Examples 5 and 6 and Comparatives 5 and 6 gave the results shown in Table 2.

TABLE 2

| | | | | pH after culture | | Cell count afer culture | |
|---|---|---|---|---|---|---|---|
| | | | | 8 h | | (cfu/ml)(TOS) | |
| | | | pH | | vs | 8 h | |
| | | Base | before | | before | Cell | Survival |
| | Spirulina | solution | culture | pH | culture | count | rate |
| Example 5 | ∘ | tap water | 6.78 | 4.82 | −1.96 | 5.6E+07 | 56% |
| Example 6 | ∘ | cow milk | 6.75 | 6.02 | −0.73 | 5.5E+08 | 545% |
| Comparative 5 | x | tap water | 8.37 | 7.63 | −0.74 | 3.0E+07 | 30% |
| Comparative 6 | x | cow milk | 6.65 | 6.46 | −0.19 | 9.5E+07 | 95% |

As shown in Table 2, also in lactic acid bacteria, similarly to the aforementioned Experimental Example 1, culture in the presence of Spirulina was effective in bacterial growth promotion and increase in survivability. Even 8 hours after culture or later, similar effects were observed.

### Experimental Example 3

In Experimental Example 3, effects of simultaneous use of Spirulina and proteins and/or saccharides on Bifidobacterium bacteria growth property were investigated.

As Bifidobacterium bacteria, Bifidobacterium longum was employed.

### (1) Preparation of Each Experimental Material

### (1-1) Bifidobacterium Bacteria Powder Preparation

By a method similar to that in Experimental Example 1, Bifidobacterium bacteria powder was prepared.

### (1-2) Spirulina Solution Preparation

### Examples 7 and 8

3 g of Spirulina powder and proteins (casein or whey protein) were dissolved in 100 mL of tap water to prepare a Spirulina solution having a protein concentration of 3% by weight. This Spirulina solution was subjected to autoclave pasteurization at 90° C. for 10 minutes to achieve sterilization.

### Examples 9 and 10

3 g of Spirulina powder and saccharides (lactose or glucose) were dissolved in 100 mL of tap water to prepare a Spirulina solution having a saccharide concentration of 5% by weight. This Spirulina solution was subjected to autoclave pasteurization at 90° C. for 10 minutes to achieve sterilization.

### Example 11

3 g of Spirulina powder and skim milk powder were dissolved in 100 mL of tap water to prepare a Spirulina solution having a skim milk powder concentration of 10% by weight. This Spirulina solution was subjected to autoclave pasteurization at 90° C. for 10 minutes to achieve sterilization.

### (2) Experimental Method

To 10 mL of each Spirulina solution placed in a test tube (sterilized), Bifidobacterium bacteria powder diluted with physiological saline at a cell count per 10 mL of 1.0×10⁹ cfu (1.0×10⁸ cfu/mL) was inoculated and cultured at 37° C. for 8 hours and 16 hours. After culturing, the cell count of the cultured material (cfu/mL) and pH were measured, compared with the cell count and pH before culturing, and effects of Spirulina, proteins, and saccharides on Bifidobacterium bacteria growth property were evaluated. Also for comparison, the aforementioned Spirulina solution was replaced by an aqueous solution having a protein concentration of 3% by weight obtained by dissolving protein (casein or whey protein) each in tap water (Comparatives 7 and 8), an aqueous solution having a saccharide concentration of 5% by weight obtained by dissolving saccharide (lactose or glucose) each in tap water (Comparatives 9 and 10), and an aqueous solution having a skim milk powder concentration of 10% by weight obtained by dissolving skim milk powder in tap water (Comparative 11), and the experiment was conducted similarly.

### (3) Results of Experiment

Examples 7 to 11 and Comparatives 7 to 11 gave the results shown in Table 3.

TABLE 3

| | | | | | pH after culture | | | | Cell count after culture | | | |
|---|---|---|---|---|---|---|---|---|---|---|---|---|
| | | | | | 8 h | | 16 h | | (cfu/ml)(TOS) | | | |
| | | | Added | pH | | vs | | vs | 8 h | | 16 h | |
| | | Base | ingredients | before | | before | | before | Cell | Survival | Cell | Survival |
| | Spirulina | solution | (concentration) | culture | pH | culture | pH | culture | count | rate | count | rate |
| Example 7 | ∘ | tap water | Casein (3%) | 6.8 | 6.67 | −0.13 | 6.29 | −0.51 | 7.3E+07 | 73% | 7.6E+07 | 76% |
| Example 8 | ∘ | tap water | Whey (3%) | 6.82 | 5.49 | −1.33 | 5.37 | −1.45 | 2.8E+08 | 281% | 3.1E+08 | 308% |
| Example 9 | ∘ | tap water | Lactose (5%) | 6.91 | 4.70 | −2.21 | 4.17 | −2.74 | 3.3E+08 | 330% | 4.0E+08 | 395% |
| Example 10 | ∘ | tap water | Glucose (5%) | 6.96 | 4.60 | −2.36 | 4.07 | −2.89 | 6.1E+08 | 605% | 5.9E+08 | 590% |
| Example 11 | ∘ | tap water | Skim milk | 6.76 | 5.18 | −1.58 | 4.49 | −2.27 | 1.1E+09 | 1065% | 5.1E+09 | 5100% |
| | | | powder (10%) | | | | | | | | | |
| Comparative 7 | x | tap water | Casein (3%) | 6.86 | 6.69 | −0.17 | 6.69 | −0.17 | 4.7E+07 | 47% | 2.2E+07 | 22 |
| Comparative 8 | x | tap water | Whey (3%) | 6.82 | 6.25 | −0.57 | 5.35 | −1.47 | 7.0E+07 | 70% | 1.0E+08 | 101% |
| Comparative 9 | x | tap water | Lactose (5%) | 7.58 | 6.16 | −1.42 | 5.39 | −2.19 | 1.6E+07 | 16% | 8.7E+05 | 0.9% |
| Comparative 10 | x | tap water | Glucose (5%) | 7.49 | 5.32 | −2.17 | 4.99 | −2.50 | 1.1E+07 | 11% | 2.9E+05 | 0.3% |
| Comparative 11 | x | tap water | Skim milk | 6.62 | 6.26 | −0.36 | 5.89 | −0.73 | 1.0E+08 | 102% | 4.6E+07 | 46% |
| | | | powder (10%) | | | | | | | | | |

As shown in Table 3, culture of Bifidobacterium bacteria in the presence of whey, lactose (disaccharide), or glucose (monosaccharide) resulted in suppressed bacterial growth which leaded to a reduced survivability (Comparatives 8 to 10), while combination with Spirulina served to promote the bacterial growth of Bifidobacterium bacteria, which resulted in a far more substantially increased cell count as shown in Examples 8 to 10. Combination of casein with Spirulina also served to increase the survivability of Bifidobacterium bacteria. Furthermore, combination of skim milk powder with Spirulina also served to further promote the growth of Bifidobacterium bacteria, resulting in a further increased cell count. Since lactose among the aforementioned saccharides is a major saccharide which constitutes cow milk, the increased survivability of Bifidobacterium bacteria caused by cow milk indicated in Comparatives 2 and 4 of the aforementioned Experimental Example 1 is considered to be attributable to ingredients contained in cow milk which are other than fats and saccharides.

### Experimental Example 4

In Experimental Example 4, effects of Spirulina on Bifidobacterium bacteria growth property when using soy milk instead of cow milk were investigated.

As Bifidobacterium bacteria, Bifidobacterium longum was employed.

### (1) Preparation of Each Experimental Material

### (1-1) Bifidobacterium Bacteria Powder/Lactic Acid Bacteria Powder Preparation

By a method similar to that in Experimental Example 1, Bifidobacterium bacteria powder was prepared.

### (1-2) Spirulina Solution Preparation

### Example 12

3 g of Spirulina powder was dissolved in 100 mL of soy milk to prepare Spirulina solution. This Spirulina solution was subjected to autoclave pasteurization at 90° C. for 10 minutes to achieve sterilization.

### Example 13

3 g of Spirulina powder and saccharides (lactose) were dissolved in 100 mL of soy milk to prepare a Spirulina solution having a saccharide concentration of 5% by weight. This Spirulina solution was subjected to autoclave pasteurization at 90° C. for 10 minutes to achieve sterilization.

### (2) Experimental Method

To 10 mL of each Spirulina solution placed in a test tube (sterilized), Bifidobacterium bacteria powder diluted with physiological saline at a cell count per 10 mL of 1.0×10⁹ cfu (1.0×10⁸ cfu/mL) was inoculated and cultured at 37° C. for 8 hours and 16 hours. After culturing, the cell count of the cultured material (cfu/mL) and pH were measured, compared with the cell count and pH before culturing, and effects of soy milk and saccharides on the growth property of Bifidobacterium bacteria or lactic acid bacteria were evaluated. Also for comparison, the aforementioned Spirulina solution was replaced by soy milk (Comparative 12) and an aqueous solution of saccharide (lactose) dissolved in soy milk having a saccharide concentration of 5% by weight (Comparative 13), and the experiment was conducted similarly.

### (3) Results of Experiment

Examples 12 and 13 and Comparatives 12 and 13 gave the results shown in Table 4.

TABLE 4

| | | | | | pH after culture | | | | Cell count after culture | | | |
|---|---|---|---|---|---|---|---|---|---|---|---|---|
| | | | | | 8 h | | 16 h | | (cfu/ml)(TOS) | | | |
| | | | Added | pH | | vs | | vs | 8 h | | 16 h | |
| | | Base | ingredients | before | | before | | before | Cell | Survival | Cell | Survival |
| | Spirulina | solution | (concentration) | culture | pH | culture | pH | culture | count | rate | count | rate |
| Example 12 | ∘ | Soy milk | x | 6.52 | 6.30 | −0.22 | 5.52 | −1.00 | 2.3E+08 | 226% | 1.2E+09 | 1150% |
| Example 13 | ∘ | Soy milk | Lactose (5%) | 6.50 | 5.10 | −1.40 | 4.64 | −1.86 | 1.4E+09 | 1440% | 1.9E+09 | 1905% |
| Comparative 12 | x | Soy milk | x | 6.55 | 6.42 | −0.13 | 6.01 | −0.54 | 1.0E+08 | 100% | 2.9E+08 | 286% |
| Comparative 13 | x | Soy milk | Lactose (5%) | 6.50 | 5.38 | −1.12 | 4.93 | −1.57 | 4.4E+08 | 440% | 2.5E+08 | 255% |

Based on the comparison between Example 12 and Comparative 12, it was proven that even when using soy milk as a base, use of Spirulina resulted in an increased survivability of Bifidobacterium bacteria. Also as indicated in Comparative 13, it was proven that use of lactose in the soy milk base served to promote the growth of Bifidobacterium bacteria, while simultaneous use of Spirulina resulted in a substantially promoted bacterial growth as indicated in Example 13, thereby increasing survivability substantially.

### Experimental Example 5

In Experimental Example 5, effects of Spirulina concentration on Bifidobacterium bacteria growth property were investigated.

As Bifidobacterium bacteria, Bifidobacterium longum was employed.

### (1) Preparation of Each Experimental Material

### (1-1) Bifidobacterium Bacteria Powder Preparation

By a method similar to that in Experimental Example 1, Bifidobacterium bacteria powder was prepared.

### (1-2) Spirulina Solution Preparation

Spirulina powder was dissolved in 100 mL of cow milk at each concentration indicated in Table 5 shown below to prepare each Spirulina solution. This Spirulina solution was subjected to autoclave pasteurization at 90° C. for 10 minutes to achieve sterilization.

### (2) Experimental Method

To 10 mL of each Spirulina solution placed in a test tube (sterilized), Bifidobacterium bacteria powder diluted with physiological saline at a cell count per 10 mL of 1.0×10⁹ cfu (1.0×10⁸ cfu/mL) was inoculated and cultured at 37° C. for 8 hours and 16 hours. After culturing, the cell count of the cultured material (cfu/mL) and pH were measured, compared with the cell count and pH before culturing, and effects of Spirulina concentration on Bifidobacterium bacteria growth property were evaluated.

### (3) Results of Experiment

Examples 14 to 18 gave the results shown in Table 5.

TABLE 5

| | | | | pH after culture | | | | Cell count afer culture | | | |
|---|---|---|---|---|---|---|---|---|---|---|---|
| | | | | 8 h | | 16 h | | (cfu/ml)(TOS) | | | |
| | Spirulina | | pH | | vs | | vs | 8 h | | 16 h | |
| | concentration | Base | before | | before | | before | Cell | Survival | Cell | Survival |
| | (% by weight) | solution | culture | pH | culture | pH | culture | count | rate | count | rate |
| Example 14 | 6% | cow milk | 6.61 | 4.70 | −1.91 | 4.28 | −2.33 | 2.5E+09 | 2455% | 2.7E+09 | 2695% |
| Example 15 | 3% | cow milk | 6.77 | 4.74 | −2.03 | 4.14 | −2.63 | 1.6E+09 | 1605% | 3.7E+09 | 3650% |
| Example 16 | 1.5% | cow milk | 6.80 | 4.93 | −1.87 | 4.17 | −2.63 | 1.6E+09 | 1605% | 2.1E+09 | 2065% |
| Example 17 | 0.3% | cow milk | 6.70 | 5.65 | −1.05 | 4.80 | −1.90 | 4.7E+08 | 465% | 7.0E+08 | 700% |
| Example 18 | 0.03% | cow milk | 6.68 | 6.10 | −0.58 | 5.62 | −1.06 | 1.1E+08 | 106% | 7.3E+07 | 73% |

As shown in Table 5, it was proven that the growth promotion effect on Bifidobacterium bacteria was raised in a Spirulina concentration-dependent manner. In Example 18, it was considered that the growth peaked during a period 8 hours to 16 hours after culture.

### Experimental Example 6

In Experimental Example 6, tap water was replaced by phosphate buffer, and effects of Spirulina and saccharides on Bifidobacterium bacteria growth property were investigated.

As Bifidobacterium bacteria, Bifidobacterium longum was employed.

### (1) Preparation of Each Experimental Material

### (1-1) Bifidobacterium Bacteria Powder Preparation

By a method similar to that in Experimental Example 1, Bifidobacterium bacteria powder was prepared.

### (1-2) Phosphate Buffer Preparation

A 0.2M sodium dihydrogenphosphate solution and a 0.2M disodium hydrogenphosphate solution were mixed and pH was adjusted at 7.

### (1-3) Spirulina-Phosphate Buffer Solution Preparation

### Example 21

3 g of Spirulina powder was dissolved in 100 mL of phosphate buffer solution to prepare Spirulina-phosphate buffer solution. This Spirulina-phosphate buffer solution was subjected to autoclave pasteurization at 90° C. for 10 minutes to achieve sterilization.

### Examples 19 and 20

3 g of Spirulina powder and saccharides (lactose) were dissolved in 100 mL of phosphate buffer solution to prepare Spirulina-phosphate buffer solution having a saccharide concentration of 5% by weight. This Spirulina-phosphate buffer solution was subjected to autoclave pasteurization at 90° C. for 10 minutes to achieve sterilization.

### (2) Experimental Method

To 10 mL of each Spirulina-phosphate buffer solution (sterilized) place in a test tube, Bifidobacterium bacteria powder diluted with physiological saline at a cell count per 10 mL of 1.0×10⁹ cfu (1.0×10⁸ cfu/mL) was inoculated and cultured at 37° C. for 8 hours and 16 hours. After culturing, the cell count of the cultured material (cfu/mL) and pH were measured, compared with the cell count and pH before culturing, and effects of Spirulina-phosphate buffer solution and Spirulina-phosphate buffer solution+saccharide on Bifidobacterium bacteria growth property were evaluated. Also for comparison, the aforementioned Spirulina-phosphate buffer solution was replaced by a phosphate buffer solution (Comparative 14) and a phosphate buffer solution having a saccharide concentration of 5% by weight obtained by dissolving saccharide (lactose) in the phosphate buffer solution (Comparative 15), and the experiment was conducted similarly.

### (3) Results of Experiment

Examples 19 and 20 and Comparatives 14 and 15 gave the results shown in Table 6.

TABLE 6

| | | | | | pH after culture | | | | Cell count after culture | | | |
|---|---|---|---|---|---|---|---|---|---|---|---|---|
| | | | | | 8 h | | 16 h | | (cfu/ml)(TOS) | | | |
| | | | Added | pH | | vs | | vs | 8 h | | 16 h | |
| | | Base | ingredients | before | | before | | before | Cell | Survival | Cell | Survival |
| | Spirulina | solution | (concentration) | culture | pH | culture | pH | culture | count | rate | count | rate |
| Example 19 | ∘ | Phosphate | x | 6.93 | 6.95 | 0.02 | 6.91 | −0.02 | 7.9E+07 | 79% | 5.1E+07 | 51% |
| | | buffer | | | | | | | | | | |
| Example 20 | ∘ | Phosphate | Lactose (5%) | 6.89 | 6.50 | −0.39 | 5.58 | −1.31 | 3.6E+08 | 360% | 9.3E+08 | 930% |
| | | buffer | | | | | | | | | | |
| Comparative 14 | x | Phosphate | x | 6.95 | 6.93 | −0.02 | 6.94 | −0.01 | 3.9E+07 | 39% | 9.6E+06 | 10% |
| | | buffer | | | | | | | | | | |
| Comparative 15 | x | Phosphate | Lactose (5%) | 6.94 | 6.92 | −0.02 | 6.91 | −0.03 | 8.0E+06 | 8% | 2.0E+05 | 0.2% |
| | | buffer | | | | | | | | | | |

As indicated in Comparatives 14 and 15, it was proven that Bifidobacterium bacteria cultured in the presence of the phosphate buffer still suffered from a low survivability which was however somewhat better than in the presence of tap water, and additional simultaneous use of a saccharide (lactose) resulted in a further reduction in the survivability. On the other hand, it was proven that, when Spirulina was further combined, the survivability of Bifidobacterium bacteria was increased (Example 19) and simultaneous use of Spirulina and lactose served to promote the growth of Bifidobacterium bacteria, thereby increasing the cell count substantially (Example 20).

It is also possible that the present technology implements the following constitutions.

(1) A growth-promoting and/or decrease-suppressing agent for Bifidobacterium bacteria and/or lactic acid bacteria containing Arthrospira blue-green algae and/or Spirulina blue-green algae as an active ingredient.
(2) The growth-promoting and/or decrease-suppressing agent according to (1) which comprises the aforementioned Arthrospira blue-green algae and/or the aforementioned Spirulina blue-green algae in one or two or more forms selected from algae body as being cultured in a liquid medium or wet algae body, algae body debris, and dried materials thereof (including milled powder) as well as solvent extracts of, algae body or wet algae body.
(3) The growth-promoting and/or decrease-suppressing agent according to (1) or (2) further including at least one selected from the group consisting of proteins and saccharides.
(4) The growth-promoting and/or decrease-suppressing agent according to (3) wherein the aforementioned protein is milk protein and/or plant protein.
(5) The growth-promoting and/or decrease-suppressing agent according to (3) or (4) wherein the aforementioned saccharides are at least one selected from the group consisting of monosaccharides, disaccharides, and oligosaccharides.
(6) An oral composition including Arthrospira blue-green algae and/or Spirulina blue-green algae and at least one type of bacteria selected from the group consisting of Bifidobacterium bacteria and lactic acid bacteria.
(7) The oral composition according to (6) which comprises the aforementioned Arthrospira blue-green algae and/or the aforementioned Spirulina blue-green algae in one or two or more forms selected from algae body as being cultured in a liquid medium or wet algae body, algae body debris, and dried materials thereof (including milled powder) as well as solvent extracts of algae body or wet algae body.
(8) The oral composition according to (6) or (7) further including at least one selected from the group consisting of proteins and saccharides.
(9) The oral composition according to (8) wherein the aforementioned protein is milk protein and/or plant protein.
(10) The oral composition according to (8) or (9) wherein the aforementioned saccharides are at least one selected from the group consisting of monosaccharides, disaccharides, and oligosaccharides.
(11) The oral composition according to any one of (6) to (10) which is used for intestinal flora improvement, immunoregulation, prophylaxis and/or treatment of diarrhea, constipation, obesity, or inflammatory bowel disease.
(12) A method for promoting growth of Bifidobacterium bacteria and/or lactic acid bacteria and/or suppressing the decrease including a step for allowing at least one type of bacteria selected from the group consisting of Bifidobacterium bacteria and lactic acid bacteria to coexist with Arthrospira blue-green algae and/or Spirulina blue-green algae.
(13) The method for promoting growth and/or suppressing decrease according to (12) which comprises the aforementioned Arthrospira blue-green algae and/or the aforementioned Spirulina blue-green algae in one or two or more forms selected from algae body as being cultured in a liquid medium or wet algae body, algae body debris, and dried materials thereof (including milled powder) as well as solvent extracts of algae body or wet algae body.
(14) The method for promoting growth and/or suppressing decrease according to (12) or (13) which further allows at least one selected from the group consisting of proteins and saccharides to coexist.
(15) The method for promoting growth and/or suppressing decrease according to (14) wherein the aforementioned protein is milk protein and/or plant protein.
(16) The method for promoting growth and/or suppressing decrease according to (14) or (15) wherein the aforementioned saccharides are at least one selected from the group consisting of monosaccharides, disaccharides, and oligosaccharides.
(17) Use of Arthrospira blue-green algae and/or Spirulina blue-green algae for an agent for promoting the growth of, and/or suppressing a decrease in, Bifidobacterium bacteria and/or lactic acid bacteria.
(18) Use of Arthrospira blue-green algae and/or Spirulina blue-green algae for an oral composition for Bifidobacterium bacteria and/or lactic acid bacteria growth promotion and/or decrease suppression.
(19) Use according to (18) used in intestinal flora improvement, immunoregulation, prophylaxis and/or treatment of diarrhea, constipation, obesity, or inflammatory bowel disease.
(20) Use of Arthrospira blue-green algae and/or Spirulina blue-green algae for production of an agent for promoting the growth of, and/or suppressing a decrease in, Bifidobacterium bacteria and/or lactic acid bacteria.
(21) Use of Arthrospira blue-green algae and/or Spirulina blue-green algae for production of an oral composition for Bifidobacterium bacteria and/or lactic acid bacteria growth promotion and/or decrease suppression.
(22) Use of Arthrospira blue-green algae and/or Spirulina blue-green algae into an agent for promoting the growth of, and/or suppressing a decrease in, Bifidobacterium bacteria and/or lactic acid bacteria.
(23) Use of Arthrospira blue-green algae and/or Spirulina blue-green algae into an oral composition for Bifidobacterium bacteria and/or lactic acid bacteria growth promotion and/or decrease suppression.
(24) Use according to (23) used in intestinal flora improvement, immunoregulation, prophylaxis and/or treatment of diarrhea, constipation, obesity, or inflammatory bowel disease.
(25) A method for promoting growth of Bifidobacterium bacteria and/or lactic acid bacteria and/or suppressing the decrease including administration of Arthrospira blue-green algae and/or Spirulina blue-green algae to applicable subjects.
(26) A method for promoting growth of Bifidobacterium bacteria and/or lactic acid bacteria and/or suppressing the decrease including administration of the growth-promoting and/or decrease-suppressing agent according to (1) to applicable subjects.
(27) A method for intestinal flora improvement using the method according to (25) or (26).
(28) An immunoregulation method using the method according to (25) or (26).
(29) A method for prophylaxis and/or treatment of diarrhea, constipation, obesity, or inflammatory bowel disease using the method according to (25) or (26).

1. A growth-promoting and/or decrease-suppressing agent for Bifidobacterium bacteria and/or lactic acid bacteria containing Arthrospira blue-green algae and/or Spirulina blue-green algae as an active ingredient.

2. The growth-promoting and/or decrease-suppressing agent according to claim 1 further comprising at least one selected from the group consisting of proteins and saccharides.

3. The growth-promoting and/or decrease-suppressing agent according to claim 2 wherein the aforementioned protein is milk protein and/or plant protein.

4. The growth-promoting and/or decrease-suppressing agent according to claim 2 wherein the aforementioned saccharides are at least one selected from the group consisting of monosaccharides, disaccharides, and oligosaccharides.

5. An oral composition comprising Arthrospira blue-green algae and/or Spirulina blue-green algae, and at least one type of bacteria selected from the group consisting of Bifidobacterium bacteria and lactic acid bacteria.

6. The oral composition according to claim 5 further comprising at least one selected from the group consisting of proteins and saccharides.

7. The oral composition according to claim 6 wherein the aforementioned protein is milk protein and/or plant protein.

8. The oral composition according to claim 6 wherein the aforementioned saccharides are at least one selected from the group consisting of monosaccharides, disaccharides, and oligosaccharides.

9. The oral composition according to claim 5 used in intestinal flora improvement, immunoregulation, prophylaxis and/or treatment of diarrhea, constipation, obesity, or inflammatory bowel disease.

10. A method for promoting growth of Bifidobacterium bacteria and/or lactic acid bacteria and/or suppressing the decrease comprising a step for allowing at least one type of bacteria selected from the group consisting of Bifidobacterium bacteria and lactic acid bacteria to coexist with Arthrospira blue-green algae and/or Spirulina blue-green algae.

11. The method for promoting growth and/or suppressing decrease according to claim 10 which further allows at least one selected from the group consisting of proteins and saccharides to coexist.

12. The method for promoting growth and/or suppressing decrease according to claim 11 wherein the aforementioned protein is milk protein and/or plant protein.

13. The method for promoting growth and/or suppressing decrease according to claim 11 wherein the aforementioned saccharides are at least one selected from the group consisting of monosaccharides, disaccharides, and oligosaccharides.
